(19) United States
(12) Patent Application Publication
(10) Pub. No.: US 2026/0114922 A1
(43) Pub. Date: Apr. 30, 2026

(54) MEDICAL DEVICES AND RELATED METHODS FOR CUTTING OR CAUTERIZING TISSUE

(71) Applicant: Boston Scientific Scimed, Inc., Maple Grove, MN (US)

(72) Inventors: Gonzalo Jose SAENZ VILLALOBOS, Alajuela (CR); Juan Pablo ORTIZ GARCIA, Heredia (CR); Cristopher OVIEDO MIRANDA, Alajuela (CR); Katherin NARVAEZ NUÑEZ, Cartago (CR); Barry WEITZNER, Great Barrington, MA (US); Dennis Brian HUBBARD, JR., Lancaster, MA (US)

(73) Assignee: Boston Scientific Scimed, Inc., Maple Grove, MN (US)

(21) Appl. No.: 19/367,151

(22) Filed: Oct. 23, 2025

Related U.S. Application Data
(60) Provisional application No. 63/712,593, filed on Oct. 28, 2024.

(51) Int. Cl. A61B 18/16; A61B 18/00; A61B 18/14

(52) U.S. Cl. CPC A61B 18/16; A61B 2018/00011; A61B 2018/00077; A61B 2018/00083; A61B 2018/00178; A61B 2018/00595; A61B 2018/00601; A61B 2018/00607; A61B 2018/1412; A61B 2018/1435; A61B 2018/1467

(57) ABSTRACT

A medical device delivers energy and/or fluid. The medical device includes a handle. The handle includes one or more electrical connections configured to electrically connect the medical device to an energy source, and a fluid port configured to receive fluid from a fluid source. The medical device further includes a shaft. The shaft includes a lumen for transmitting the fluid from the fluid source and one or more conductors for transmitting energy from the energy source. The one or more conductors include a first conductor, a second conductor, and a third conductor. The medical device includes a first electrode at a distal end of the shaft. The first electrode is electrically coupled to the first conductor to deliver energy from the energy source to the first electrode. The first electrode includes a lumen having a distal opening for delivering fluid.

### CROSS-REFERENCE TO RELATED APPLICATIONS

This application claims the benefit of priority to U.S. Provisional Application No. 63/712,593, filed on Oct. 28, 2024, which is incorporated by reference herein in its entirety.

### TECHNICAL FIELD

Aspects of this disclosure generally relate to medical devices and related methods for cutting or cauterizing tissue. In particular, aspects of this disclosure relate to medical devices and related methods configured for the treatment of tissue by delivering electrical energy to tissue and/or injecting fluid into tissue with an electrode having a tissue coagulating tip.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ, a gland, or other tissue to treat tumors, infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, etc. In particular, such procedures may be carried out by inserting an insertion device into a subject's body through a surgical incision, or via a natural anatomical orifice (e.g., mouth, vagina, or rectum), and performing the procedure or operation at a target site with an auxiliary device inserted through the insertion device.

At times, during a medical procedure, a user may use an injection needle and an energy delivery device for purposes of raising, separating, flushing, cutting, dissecting, ablating, marking, coagulating, cauterizing, or otherwise treating and/or manipulating tissue. The injection and energy delivery may be performed separately. For example, in order to deliver energy to the tissue, the user may be required to remove the injection needle from the insertion device and deliver the energy delivery device through the insertion device to the tissue being targeted, and vice versa. During the procedure, the user may alternate using the injection needle and the energy delivery device, and exchanging devices may increase the duration and risks of the medical procedure.

One method for sealing potential hemorrhaging or bleeding areas is via electrical coagulation of the target site. An electro-coagulation device may be inserted through an endoscope to the target site. In some cases, however, additional treatment of the tissue (e.g., ablation, cutting) may be necessary to treat the target site so that physicians may be required to use different devices to treat the target site as desired. The exchange of different devices through the endoscope may be time consuming, may increase the risk to the patient and may also increase patient discomfort. The devices and methods of the current disclosure may rectify one or more of the deficiencies described above or address other aspects of the art.

### SUMMARY

Examples of this disclosure relate to, among other things, medical devices configured for treating tissue by delivering electrical energy to the tissue for both cutting and coagulation, and configured for delivering fluid into and/or under the tissue. Each of the examples disclosed herein may include one or more of the features described in connection with any of the other disclosed examples.

In an example, a medical device may deliver energy and/or fluid. The medical device may include a handle. The handle may include one or more electrical connections configured to electrically connect the medical device to an energy source. The medical device may also include a fluid port configured to receive fluid from a fluid source. The medical device may further include a shaft. The shaft may include a lumen for transmitting the fluid from the fluid source and one or more conductors for transmitting energy from the energy source. The one or more conductors may include a first conductor, a second conductor, and a third conductor. The medical device may include a first electrode at a distal end of the shaft. The first electrode may be electrically coupled to the first conductor to deliver energy from the energy source to the first electrode. The first electrode may include a lumen having a distal opening for delivering fluid from the fluid source to the distal opening. A second electrode and a third electrode may be disposed on an outer surface of the distal end of the shaft. The second electrode may be electrically coupled to the second conductor. The third electrode may be electrically coupled to the third conductor to deliver energy from the energy source to the third electrode.

Any of the devices disclosed herein may include any of the following features, additionally or alternatively, in any combination. The medical device may include a hub. The hub may include a first electrical connection of the one or more electrical connections. The shaft may include a sheath. The sheath may define the lumen. The medical device may include a ground configured to be coupled to a patient. The ground may be electrically coupled to the first conductor. The second conductor and the third conductor may have opposite polarities. The hub may be coupled to the fluid port. The handle may further include a handle body that defines a channel. The hub may be movably coupled to the handle body. The hub may be configured to move in the channel. The medical device may further include a first actuator electrically coupled to the second conductor and the third conductor. The medical device may also include a second actuator electrically coupled to the first conductor and third conductor. In a first configuration of the first actuator and the second actuator, the energy source may be electrically connected to the first electrode, and the energy source may be not electrically connected to the second electrode and the third electrode.

The second conductor and the third conductor may include, respectively, a first electrical connection of the one or more electrical connections and a second electrical connection of the one or more electrical connections. The first conductor may have an opposite polarity of the second conductor and the third conductor. In a second configuration of the first actuator and the second actuator, the energy source may not be electrically connected to the first electrode, and the energy source may be electrically connected to the second electrode and the third electrode.

The second conductor and the third conductor may have opposite polarities. The medical device may further include an actuator electrically coupled to the first conductor and the third conductor. In a first configuration of the actuator, the energy source may be electrically connected to the first electrode, and the energy source may not be electrically connected to the second electrode and the third electrode. In a second configuration of the actuator, the energy source may not be electrically connected to the first electrode, and the energy source may be electrically connected to the second electrode and the third electrode.

The second conductor and the third conductor may include, respectively, a first electrical connection of the one or more electrical connections and a second electrical connection of the one or more electrical connections. The energy source may be a first energy source. The one or more conductors may further include a fourth conductor. The one or more electrical connections may include the first conductor, the second conductor, the third conductor, and the fourth conductor. The medical device may further include a second energy source. The second conductor and the third conductor may be electrically connected to the first energy source. The first conductor and the fourth conductor may be electrically connected to the second energy source. The first electrode may be configured to be advanced distally and retracted proximally relative to the distal end of the shaft.

The first electrode may include a shaft portion and a widened distal portion. The widened distal portion may be wider than the shaft portion. The second conductor and the third conductor may have opposite polarities. The first conductor and the fourth conductor may have opposite polarities. The second electrode and the third electrode may extend helically around the distal end of the shaft. The second electrode and the third electrode may be spaced apart.

In another example, a medical device may deliver energy. The medical device may include a handle. The handle may include one or more electrical connections configured to electrically connect the medical device to an energy source. The medical device may include a shaft. The shaft may include a lumen and a sheath. The sheath may define the lumen. The medical device may further include one or more conductors for transmitting energy from the energy source. The sheath may be electrically insulative. The medical device may include a first electrode at a distal end of the shaft. The first electrode may include a first portion electrically coupled to a first conductor of the one more conductors and may be positioned on a first side of the sheath. The first electrode may include a second portion electrically coupled to a second conductor of the one or more conductors and positioned on a second side of the sheath. The medical device may include a second electrode disposed on an outer surface of the distal end of the shaft. The second electrode may be electrically coupled to a third conductor of the one or more conductors. The medical device may include a third electrode disposed on the outer surface of the distal end of the shaft. The third electrode may be electrically coupled to a fourth conductor of the one or more conductors.

Any of the devices disclosed herein may include any of the following features, additionally or alternatively, in any combination. The first electrode may include a cutting electrode. The second electrode and the third electrode may include cauterization electrodes. The first conductor and the second conductor may have opposite polarities.

In another example, a medical device may deliver energy and/or fluid. The medical device may include a handle. The handle may include one or more electrical connections configured to electrically connect the medical device to an energy source. The handle may also include a fluid port configured to receive fluid from a fluid source. The medical device may include a shaft. The shaft may include a lumen and a sheath. The sheath may define the lumen for transmitting the fluid from the fluid source and one or more conductors for transmitting energy from the energy source. The sheath may be electrically insulative. The medical device may include first electrode at a distal end of the shaft. The first electrode may include a first portion electrically coupled to a first conductor of the one more conductors and disposed radially outward of the sheath. The first electrode may also include a second portion electrically coupled to a second conductor of the one or more conductors and disposed radially outward of the first portion. The first electrode may further include an electrical insulator disposed between the first portion and the second portion. The first electrode may include an electrode lumen extending through a portion of the first electrode. The electrode lumen may be fluidly connected to the lumen in the shaft. The electrode lumen may include a distal opening for delivering fluid from the fluid source. The medical device may include a second electrode disposed on an outer surface of the distal end of the shaft. The medical device may include a third electrode disposed on the outer surface of the distal end of the shaft. The second electrode may be electrically coupled to a third conductor of the one or more conductors. The third electrode may be electrically coupled to a fourth conductor of the one or more conductors.

Any of the devices disclosed herein may include any of the following features, additionally or alternatively, in any combination. The first portion may further include a distal protrusion. The distal protrusion may include a larger diameter than the second portion.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 illustrates an exemplary medical device, including an enlarged partially transparent view of a distal portion of the medical device, according to aspects of this disclosure.

FIG. 2 illustrates a side view of a hub of the medical device of FIG. 1, with the hub shown as partially transparent, according to aspects of this disclosure.

FIG. 3 illustrates a schematic view of a handle of the exemplary medical device of FIG. 1 and a patient, according to aspects of this disclosure.

FIG. 4 illustrates a perspective partially transparent view of a hub of a first alternative medical device, according to aspects of this disclosure.

FIG. 5 illustrates a perspective view of a handle of the first alternative exemplary medical device, according to aspects of this disclosure.

FIG. 6 illustrates a schematic view of a first alternative handle of the exemplary medical device of FIG. 1, according to aspects of this disclosure.

FIGS. 7A-7B respectively illustrate a first electrical configuration and a second electrical configuration of the first alternative handle of FIG. 6

FIG. 8A illustrates an exterior portion of a second alternative handle of the medical device of FIG. 1, and FIG. 8B illustrates a schematic view of the second alternative handle of the medical device of FIG. 1, according to aspects of this disclosure.

FIGS. 9A-9B respectively illustrates a first electrical configuration and a second electrical configuration of the second alternative handle of the medical device of FIG. 1, according to aspects of this disclosure.

FIG. 10 illustrates a cross-sectional view of a handle of a second alternative medical device, according to aspects of this disclosure.

FIG. 11 illustrates a cross-sectional view of an exemplary cutting electrode, according to aspects of this disclosure.

FIG. 12 illustrates a cross-sectional view of another exemplary cutting electrode, according to aspects of this disclosure.

### DETAILED DESCRIPTION

Examples of this disclosure include devices and methods for: facilitating and improving the efficacy, efficiency, and safety of treating and/or manipulating tissue when, for example, applying electrical energy to tissue with an electrode; delivering fluid into and/or under tissue during a medical procedure through the distal end of the electrode; and cauterizing tissue with a distal tip of the electrode. For example, aspects of this disclosure may provide a user (e.g., physician, medical technician, or other medical service provider) with the ability to apply electrical energy or heat to tissue using a medical device having an electrode, to deliver fluid into and/or under tissue, and to cauterize tissue with the same medical device. Aspects of this disclosure may provide the user with the ability to apply electrical energy or heat and deliver fluid with a reduced likelihood of damaging tissue. Some aspects of this disclosure may be used in performing an endoscopic, laparoscopic, arthroscopic, gynoscopic, thoracoscopic, cystoscopic, or other type of procedure.

Reference will now be made in detail to examples of this disclosure described above and illustrated in the accompanying drawings. Wherever possible, the same reference numbers will be used throughout the drawings to refer to the same or like parts. Each of the examples, aspects, and embodiments disclosed herein may include one or more of the features described in connection with any of the other disclosed examples, aspects, and embodiments.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

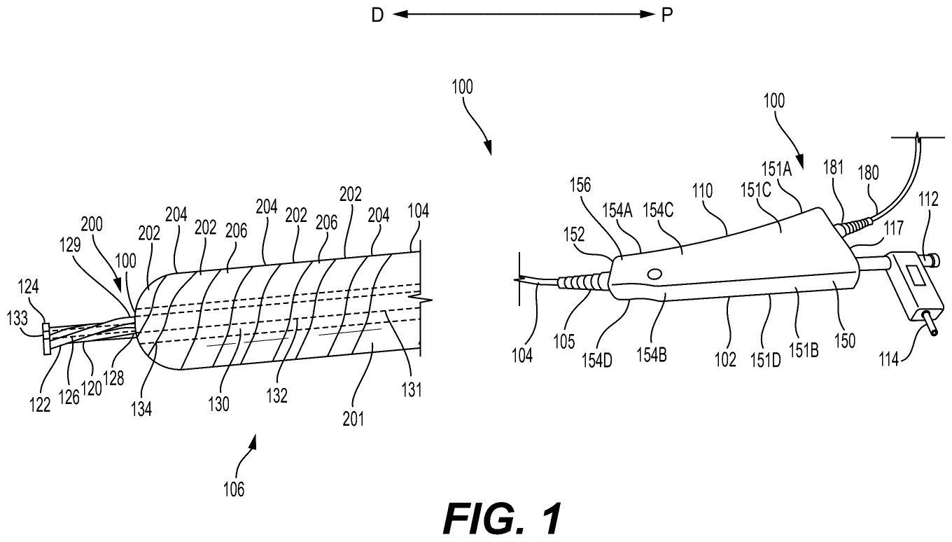

FIG. 1 depicts a medical device 100 that includes a handle 102 (FIG. 1), a shaft 104, and a distal end 106 (FIG. 1). Handle 102 may include a body 110. Handle 102 also may include or otherwise be coupled to or adjacent to a port 112 configured to receive fluid. Handle 102 may also include or otherwise be coupled to a hub 114 configured to receive electrical energy, similar to an electrical plug or socket. In some aspects, hub 114 may include port 112. Distal end 106 may include a first electrode, for example, a cutting electrode 120. Cutting electrode 120 may (i) be conductive to convey and deliver energy from hub 114 and/or (ii) convey fluid from port 112 to tissue or other material at a treatment site. Cutting electrode 120 may be movable relative to shaft 104, as described in further detail below. The distal end of shaft may include electrodes and/or electrically conductive material disposed on the distal end of the shaft configured to supply energy to tissue.

It will also be understood that, although the terms first, second, third, etc. are, in some instances, used herein to describe various elements, these elements should not be limited by these terms. These terms are only used to distinguish one element from another. For example, a first electrode could be termed a second electrode, and, similarly, a second electrode could be termed a first electrode, without departing from the scope of the various described embodiments. The first electrode and the second electrode are both electrodes, but they are not the same electrode.

As shown particularly in FIG. 1, shaft 104 may include a sheath 130 and one or more conductors, such as a wire 132. Wire 132 is illustrated and described as a single wire in FIG. 1 for ease of reference, but it will be appreciated that wire 132 may be one or more wires or cables, each of which may serve a different electrical function (described in detail below). Sheath 130 may be formed of a flexible, insulating material (e.g., plastic), such that shaft 104 may be inserted into a delivery device (e.g., an endoscopic scope, a sheath, etc.) and advanced to a target site. Sheath 130 may include any suitable number of layers, having any suitable properties. In some examples, shaft 104 may include a plurality of sheaths (e.g., inner sheaths and outer sheaths), although only one sheath 130 is shown in FIG. 1. Shaft 104 and sheath 130 may include a strain relief portion 105 at a proximal portion of shaft 104 and sheath 130 abutting a distal portion 152 of handle 102.

A distal portion 131 of sheath 130 may be connected to proximal portion 201 of a distal tip 200. Cutting electrode 120 may extend through distal portion 131 of sheath 130 and proximal portion 201 of distal tip 200, and may extend distally (e.g., movably extend distally or be retracted proximally) of or via a distal opening 128. Movement of hub 114 relative to body 110 and/or lumen 134 may control or otherwise impart movement to cutting electrode 120 (e.g., knife electrode may be extendable and retractable). Distal tip 200 may be used for performing electrohemostasis. Distal tip 200 may be formed of any suitable biocompatible and electrically insulative material, including ceramic and/or plastic.

Distal tip 200 may include a first cauterization electrode 204 and a second cauterization electrode 206 extending helically (e.g., spirally) thereabout distal tip 200. First cauterization electrode 204 and second cauterization electrode 206 may be formed of any suitable biocompatible and electrically conductive material, such as gold. First and second cauterization electrodes 204, 206 may extend about and/or along distal tip 200 in any of a variety of other configurations (e.g., longitudinal strips, circumferential bands, helical windings, etc.) First and second cauterization electrodes 204, 206 may be spaced apart from one another. First and second cauterization electrodes 204, 206 may coagulate tissue when distal tip 200 is brought into contact with a tissue surface, such as an incision created by cutting electrode 120. Distal tip 200 and/or distal end 106 may include an end cap, or may include a portion of greater rigidity as compared to intermediate and proximal portions of shaft 104. In these aspects, the greater distal rigidity may allow for greater control of the position of distal tip 200 and/or distal end 106, and the relatively less rigid intermediate and proximal portions of shaft 104 may allow for greater maneuverability through tortuous patient anatomy.

A lumen 134 may extend longitudinally through shaft 104. For example, lumen 134 may extend along an entire length of shaft 104, from handle 102 to distal end 106. As shown in FIG. 1, in particular, lumen 134 may be defined by an inner surface of sheath 130. Alternatively, where shaft 104 includes a plurality of sheaths, a surface of another sheath may define lumen 134. Lumen 134 may have a uniform width/diameter along a length of shaft 104, or a width/diameter of lumen 134 may vary along the length of shaft 104.

Wire 132 may extend through lumen 134. As described in further detail below, wire 132 may be electrically/conductively coupled to hub 114, such that energy received by hub 114 may travel along wire 132. Although a single wire 132 is depicted, it will be appreciated that device 100 may include a plurality of wires 132. Wire 132 may extend along an entire length of shaft 104 and into handle 102, as described below.

Additionally, although not shown, one or more portions of wire 132 may be coated with an insulating material. In some aspects, the insulating coating on wire 132 may help to isolate the electrically charged wire from the fluid being delivered to the treatment site through lumen 134, as discussed in further detail below.

As noted above, cutting electrode 120 may extend distally of opening 128.

Cutting electrode 120 may include a shaft 122 and a distal tip 124. Distal tip 124 may extend radially outward from a distal end of shaft 122. Distal tip 124 may have a greater diameter/width than shaft 122. In other words, distal tip 124 may be a widened distal end of cutting electrode 120. Distal tip 124 may be wider than opening 128, which may prevent cutting electrode 120 from being fully retracted within shaft 104. A lumen 126 may extend through cutting electrode 120, to a distal opening 133 on a distal face of distal tip 124. Lumen 126 of cutting electrode 120 may be in fluid communication with lumen 134 of shaft 104. For example, as shown in FIG. 1, a proximal end 129 of shaft 122 may extend proximally into lumen 134 of shaft 104. Lumen 126 of cutting electrode 120 may have a proximal opening on a proximal end of proximal end 129 of shaft 122. Alternatively, shaft 122 may include one or more side openings (not shown). Thus, fluid may flow from lumen 134 of shaft 104 into lumen 126 of cutting electrode 120. The fluid may then exit out of distal opening 133 of cutting electrode 120.

It is noted that, in other embodiments, instead of fluid being delivered through cutting electrode 120, the fluid may be delivered around cutting electrode 120. In some aspects, however, cutting electrode 120 (e.g., electrode shaft 122) may help to guide the fluid to the treatment site, for example, via capillary action.

Wire 132 may be electrically coupled/connected to cutting electrode 120 (e.g., to shaft 122). For example, wire 132 may be coupled to proximal end 129 of shaft 122 using any suitable mechanism (e.g., solder, conductive adhesive, weld, etc.). Cutting electrode 120 may be conductive, such that electrical energy traveling through wire 132 may be delivered to cutting electrode 120, thereby energizing cutting electrode 120, including shaft 122 and/or distal tip 124. Additionally, although not shown, one or more portions of cutting electrode 120 may include insulation, for example, radially surrounding one or more portions of cutting electrode 120. Cutting electrode 120 (and wire 132) may be movable relative to distal tip 200. One or more stopping mechanisms (not shown) at a proximal portion of cutting electrode 120 may prevent additional distal movement of cutting electrode 120 beyond a distalmost position.

With reference to FIG. 1, body 110 of handle 102 may have an elongated and/or curved approximately trapezoidal shape along a longitudinal axis of body 110. A proximal portion 150 of body 110 may have an approximately rectangular or square cross-sectional shape, such that proximal portion 150 may be approximately a rectangular prism. For example, proximal portion 150 may have a first wall 151A, a second wall 151B, a third wall 151C, and a fourth wall 151D. Distal portion 152 of body 110 may have a smaller cross-sectional area than proximal portion 150. A radially-inward curved surface may connect first wall 151A (located at the proximal portion 150) and top wall 154A (located as the distal portion 152). Thus, the cross-sectional area of handle 102 may decrease in the distal direction along the central longitudinal axis of handle 102 (e.g., tapering from a larger cross-section at proximal portion 150 to a smaller cross-section at distal portion 152).

At a distal portion 152 of body 110, opposing top wall 154A and bottom wall 154B of distal portion 152 body 110 (walls on opposite sides of the central longitudinal axis of body 110) may taper radially inward in a distal direction, toward a central longitudinal axis of body 110. It will be appreciated that handle 102 may be rotated, and that the terms “top” and “bottom” are used for ease of reference and refer to the view of FIG. 1. Handle 102 may also have two side walls 156 (only one of which is visible in FIG. 1) that may be planar and may extend along an entire length of body 110, along proximal portion 150 and distal portion 152.

Hub 114 may be positioned on a proximal face 117 of proximal portion 150.

Wire 132 may receive energy from hub 114. For example, hub 114 may include one or more pins or prongs to electrically connect/couple to the energy source. The energy source may be an electrocautery source, a radio frequency generator, a heating source, a current generator, a battery, etc. In some examples, hub 114 may be removably coupleable to a cord (e.g., via pins or prongs), which may transmit energy from the energy source. Hub 114 may be electrically coupled to wire 132 via any suitable connection (e.g., soldering, conductive adhesive, etc.). In some examples, wire 132 may extend from cutting electrode 120, through shaft 104, and into handle 102, to hub 114. Alternatively, intermediate wires, cables, or other conductive elements may extend between hub 114 and wire 132. A sheath 180 may enclose one or more wires that are operably and electrically connected to handle 102 at proximal face 117. Sheath 180 may include one or more wires that are operably coupled to one or more actuators of handle 102 and/or surgical tools located at distal tip 200. The wire(s) of sheath 180 may receive energy from an energy source. The energy source may be an electrocautery source, a radio frequency generator, a heating source, a current generator, etc. Sheath 180 may include a strain relief portion 181 at a distal end of sheath 180 that is proximal of proximal face 117.

In one aspect, medical device 100 may be used for monopolar electrosurgery.

For example, a return electrode may be positioned remotely from cutting electrode 120 on or otherwise adjacent to the subject. In another aspect, medical device 100 may be used for bipolar electrosurgery. In that instance, cutting electrode 120 may include an active electrode portion, and a return electrode may be provided at or near another portion of cutting electrode 120 and/or shaft 104. In one example, although not shown, two conductive elements (e.g., wires 132) may run through shaft 104. The conductive elements may be electrically isolated from each other, allowing one to conduct energy to the active electrode and the other to conduct energy from a return electrode. Although not shown, in another aspect, the energy source may be a part of handle 102 (e.g., an internal battery in handle 102).

Hub 114 may include fluid port 112. As shown in FIG. 1, fluid port 112 may be on a proximal end of handle 102. Fluid port 112 may be couplable to a fluid source (not shown) such as, for example, a syringe, a pump, a reservoir, etc., containing a fluid. Fluid port 112 may be in fluid communication with lumen 134 of shaft 104, such that fluid from fluid port 112 may flow into lumen 134. Accordingly, shaft 104 may deliver fluid to the treatment site.

In some examples, shaft 104 may extend proximally through handle 102, such that a proximal end of shaft 104 is coupled to fluid port 112. Alternatively, shaft 104 may have a proximalmost end at a distal end of handle 102, or shaft 104 may extend proximally only partially into handle 102.

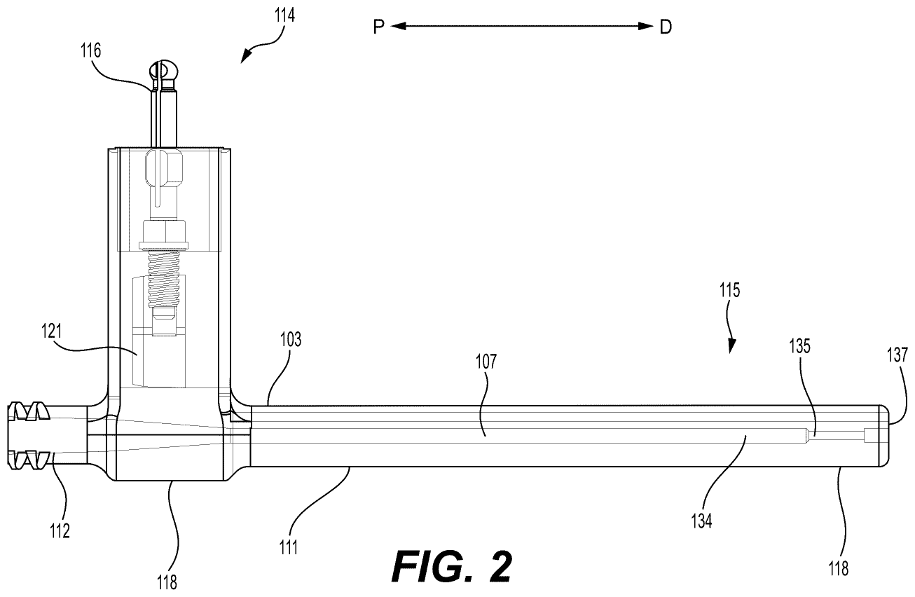

FIG. 2 illustrates a longitudinal side view of hub 114 of medical device 100 of FIG. 1. Hub 114 may be positioned on one side of proximal portion 150 (FIG. 1). Wire 132 may receive energy from hub 114. For example, hub 114 may include one or more pins or prongs to electrically connect/couple to the energy source. The energy source may be an electrocautery source, a radiofrequency generator, a heating source, a current generator, etc. In some examples, hub 114 may be removably coupleable to a cord (e.g., via pins or prongs), which may transmit energy from the energy source. Hub 114 may be electrically coupled to wire 132 via any suitable connection (e.g., soldering, conductive adhesive, etc.). In some examples, wire 132 may extend from cutting electrode 120, through shaft 104, and into handle 102, to hub 114. Alternatively, intermediate wires, cables, or other conductive elements may extend between hub 114 and wire 132. Further discussion of the electrical configurations of hub 114 are provided below.

In one example, an electrosurgical generator coupled to the handle (or within the handle) may generate receive energy in various modes, for example, radio frequency energy in a cutting mode, a coagulation mode, etc., in order for the electrode to deliver these different modes of energy to the tissue. In one aspect, the electrosurgical generator and/or the handle may include one or more knobs, dials, buttons, etc. in order to select the energy mode. Additionally, in one example, a fluid source (e.g., a saline source) coupled to the handle may provide fluid (e.g., saline) to be delivered through the electrode to the tissue and/or the target site. The fluid may be delivered at a constant rate, a pulsed rate, a user-controlled rate, etc. In these aspects, one or more of the energy delivery and/or the fluid delivery may be controlled by one or more actuators (e.g., triggers, buttons, touch screens, foot pedals, etc.).

A proximal portion 118 of hub 114 may include port 112 that may act as a point of connection with a syringe or other fluid source, through which a fluid, such as saline solution, may flow. Hub 114 may be configured to receive electrical energy, similar to an electrical plug or socket. Hub 114 may supply energy via one or more wires (e.g., wire 132) to both an electrocautery function and/or a cutting function. Hub 114 may include a 2-in-1 active cord 116, which may be electrically connected to an active cord 121. 2-in-1 active cord 116 and active cord 121 may enable bipolar cutting functionality of cutting electrode 120. Active cord 121 may be electrically connected to a wire 135, which may extend distally through a lumen 134, from a proximal portion 103 of hub 114, through a middle portion 107 of hub 114, to a distal portion 115 of hub 114. Wire 135 may be welded, crimped, or otherwise operably connected to active cord 121. Wire 135 may extend longitudinally with one or more additional wires included in handle 102 and operably connected to one or more functional aspects of distal tip 200. In the first aspect, wire 135 may be used with a monopolar configuration of cutting electrode 120.

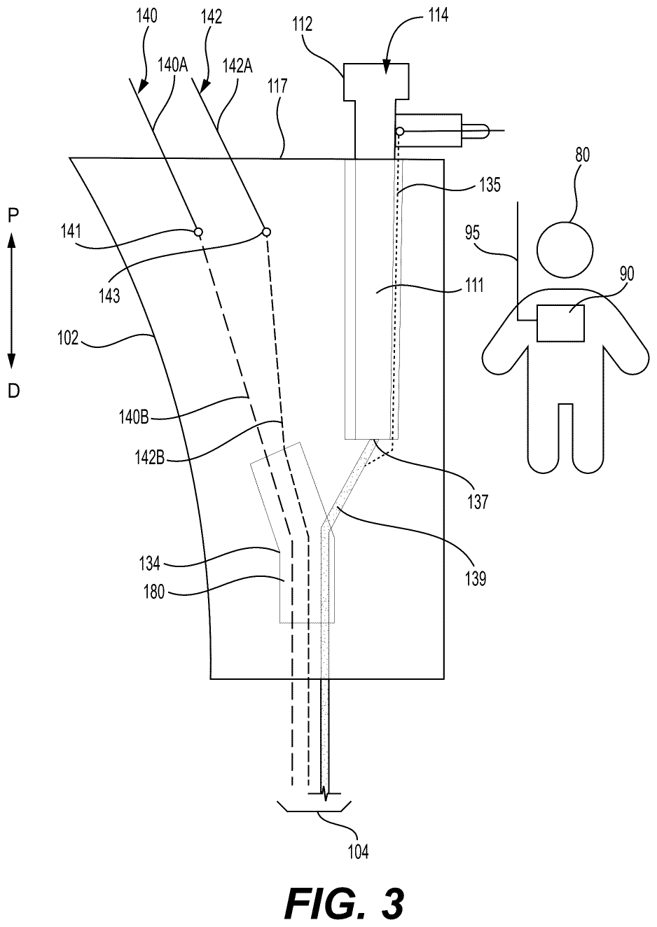

FIG. 3 illustrates a schematic view of a handle of the exemplary medical device of FIG. 1 and a patient, according to aspects of this disclosure. FIG. 3 may illustrate an arrangement of one or more electrical elements, including one or more wires. As discussed above, medical device 100 may include both cutting and cauterization functionality. To facilitate both functions, medical device 100 may have various arrangements of electrical components. In one aspect of the disclosure, the cutting function may be monopolar, and the cauterization function may be bipolar. A wire 140 and a wire 142 may extend longitudinally through handle 102 and shaft 104 to distal tip 200. Wire 140 may include a proximal portion 140A and a distal portion 140B.

Proximal portion 140A may be electrically connected and disconnected to distal portion 140B via an actuator 141. Wire 142 may include a proximal portion 142A and a distal portion 142B. Proximal portion 142A may be electrically connected and disconnected to distal portion 142B via an actuator 143. While wire 140 and wire 142 are depicted as discrete wires entering handle 102 at proximal face 117, it will be appreciated that this is only exemplary and that wires 140 and 142 may each be positioned within a single sheath, such as sheath 180. Each of wires 140 and 142 may be associated a cauterization electrode of distal tip 200. For example, wire 140 may be operably connected to cauterization electrode 204, and wire 142 may be operably connected to cauterization electrode 206, though this is only exemplary. Wires 140 and 142 may have opposite polarities. Thus, cauterization electrodes 204 and 206 (FIG. 1) may also have opposite polarities. It will be understood that for energy to flow from wire 140 to wire 142 (and vice-versa), actuators 141 and 143 may need to be in a certain configuration such that proximal portion 140A is electrically connected to distal portion 140B, and that proximal portion 142A is electrically connected to distal portion 142B, respectively.

In some aspects, wire 135 may be enclosed within a sheath 139 extending from a distal opening 137 through handle 102, through shaft 104, to distal tip 200 to operably couple to cutting electrode 120. In some aspects, incoming fluid from port 112 may also pass through sheath 139 to be delivered to distal tip 200. Thus, in some aspects, sheath 139 may include wire 135 and fluid. In some aspects, wire 135 may run outside and along sheath 139 (e.g., without being surrounded by fluid from port 112).

In the first aspect, the cutting function may be monopolar. Wire 135 may extend through a longitudinal portion 111 of hub 114 and out of opening 137. Wire 135 may be electrically charged by a source (e.g., a source electrically connected to 2-in-1 active cord 116). Due to the monopolar configuration for cutting functionality in the first aspect, a ground wire 95 may be affixed to a patient 80 via a grounding patch 90 (e.g., a return electrode). Electrical energy delivered to tissue of patient 80 via cutting electrode 120 may pass through the tissue or other portions of the treatment site or patient 80 and complete an electrical circuit with grounding patch 90 and ground wire 95 operably connected to an energy source.

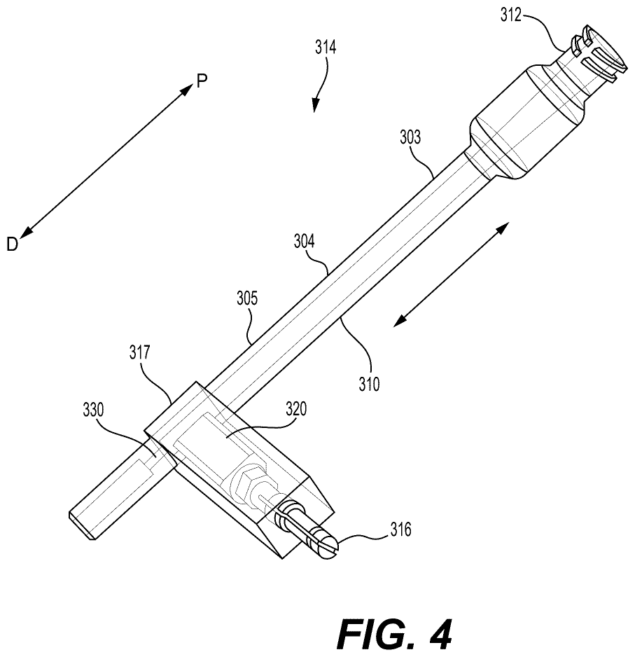

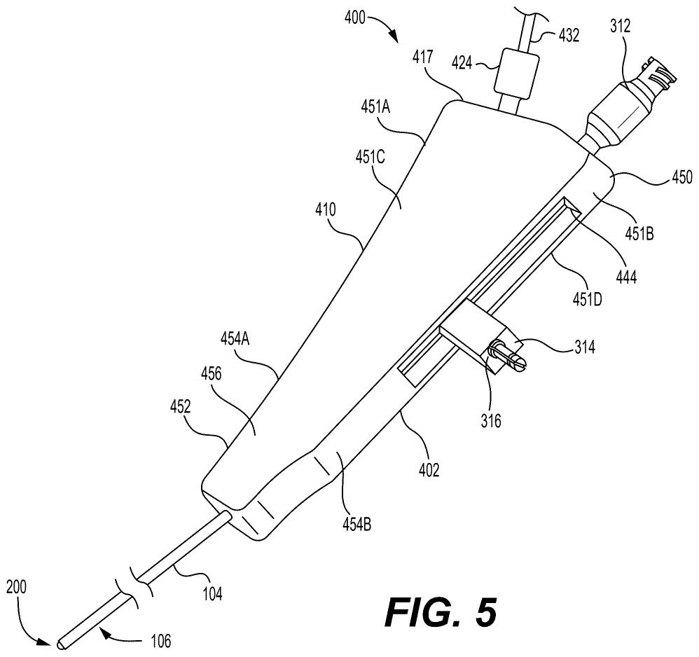

FIG. 4 illustrates a perspective partially transparent view of a hub of a first alternative medical device, according to aspects of this disclosure. Reference numerals in FIG. 4 refer to substantially similar components as FIG. 2, with “100” added to each reference numeral, unless otherwise noted. Hub 314 and a handle 402 (shown in FIG. 5) may be operably compatible with distal tip 200, as described in FIG. 1. In this aspect of the disclosure, a 2-in-1 active hub 316 and an active hub 320 may be positioned at a distal portion 305 of a longitudinal portion 310 of hub 314. Hub 314 (including 2-in-1 active hub 316, active hub 320, and port 312) may slide longitudinally relative to handle 402. Movable hub 314 may be electrically and fixedly coupled to a wire 330. Movement of hub 314 may impart movement to wire 330, which in turn may impart movement to cutting electrode 120 (e.g., extending and/or retracting cutting electrode 120). Wire 330 may be smaller in diameter than wire 135 as discussed above. Hub 314 may slide within a channel of a medical device handle, discussion of which is provided with respect to FIG. 5. Hub 314 may slide each time a knife electrode is actuated (e.g., cutting electrode 120).

FIG. 5 illustrates a handle 402 of a medical device 400, according to aspects of the disclosure. With reference to FIG. 4, body 410 of handle 402 may have an elongated and/or curved approximately trapezoidal shape along a longitudinal axis of body 410. A proximal portion 450 of body 410 may have an approximately rectangular or square cross-sectional shape, such that a cross-section of proximal portion 450 may be approximately a rectangular prism. For example, proximal portion 450 may have a first wall 451A, a second wall 451B, a third wall 451C, and a fourth wall 451D. Distal portion 452 of body 410 may have a smaller cross-sectional area than proximal portion 450. A radially-inward curved surface may connect first wall 451A and top wall 454A. Thus, the cross-sectional area of handle 402 may decrease in the distal direction along the central longitudinal axis of handle 402 (e.g., tapering from a larger cross-section at proximal portion 450 to a smaller cross-section at distal portion 452).

At a distal portion 452 of body 410, opposing top and bottom walls 454A, 454B of distal portion 452 of body 410 (e.g., walls on opposite sides of the central longitudinal axis of body 410) may taper radially inward in a distal direction, toward a central longitudinal axis of body 410. It will be appreciated that handle 402 may be rotated, and that the terms “top” and “bottom” are used for ease of reference and refer to the view depicted in FIG. 4. Handle 402 may also have two side walls 456 (only one of which is visible in FIG. 4) that may be planar and may extend along an entire length of body 410, along proximal portion 450 and distal portion 452.

Handle 402 may be operably compatible with hub 314 as described with respect to FIG. 4. Various aspects of the disclosure may encompass both handle 402 and hub 314. Reference numerals in FIG. 5 refer to substantially similar components as FIG. 1, with “300” added to each reference numeral, unless otherwise noted. Handle 402 may include a channel 444. For example, channel 444 may be disposed on third wall 451B and/or third side 454B. A port 312 of hub 314 may extend from a proximal face 417. In an example, a user may operably connect an energy source (e.g., in a monopolar configuration) to 2-in-1 active hub 314, which may be positioned at a proximal portion of channel 444. A user may slide or otherwise move hub 314 distally within channel 444, which may extend cutting electrode 120 relative to distal end 106 (and distal tip 200) of shaft 104. Additionally, one or more energy sources (not shown) may be activated to deliver energy to cutting electrode 120 via hub 314 and wire 330, for example, to cut or otherwise treat tissue or a treatment site. Furthermore, a user may slide or otherwise move hub 314 proximally within channel 444 to proximally retract cutting electrode 120 relative to distal end 106, distal tip 200, and/or shaft 104.

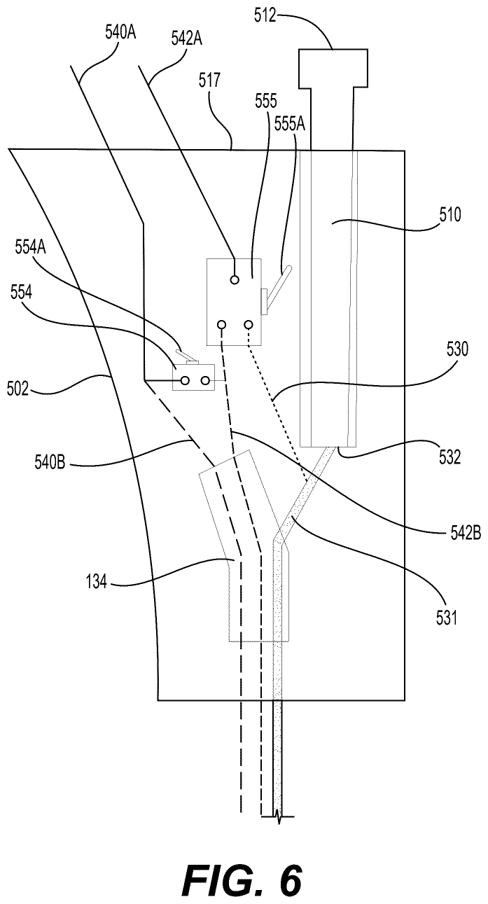

FIG. 6 illustrates a handle 502 of a medical device, according to another aspect of the disclosure. In this aspect, both the cauterization function and cutting function of the medical device 100 may use a bipolar configuration. As shown in FIG. 6, handle 502 includes a port 512, but may not include an electrical hub (e.g., a hub substantially similar to hub 114). Incoming fluid from port 512 may flow through fluid channel 510 to an opening 532, and fluid may then flow distally though sheath 531 to a distal tip (not shown).

As mentioned, both the cauterization function (e.g., cauterization electrodes 204 and 206) and the cutting function (e.g., via electrode 120) may utilize and be operably connected to a same power source. A wire 540A may be proximal of an actuator 554, and a wire 540B may be distal to an actuator 554. A wire 542A may be proximal of an actuator 555, and a wire 542B may be distal of an actuator 555. Wire 540A may be selectively electrically connected to wire 540B based on actuation of actuator 554. Wire 542A may be selectively electrically connected/disconnected to wire 542B based on actuation of actuator 555. Wire 540B may correspond and operably connect to cauterization electrode 204, and wire 542B may correspond to and operably connect to cauterization electrode 206 and cutting electrode 120, based on the configuration of actuators 554 and 555. Wires 540A/540B may have a first polarity, and wires 542A/542B may have a second, opposite polarity. A wire 530 may also extend from actuator 555. In some aspects, wire 530 may extend distally through sheath 531 to cutting electrode 120. In some aspects, wire 530 may extend distally to cutting electrode 120 outside of sheath 531.

As mentioned, handle 502 may include actuator 554 and actuator 555.

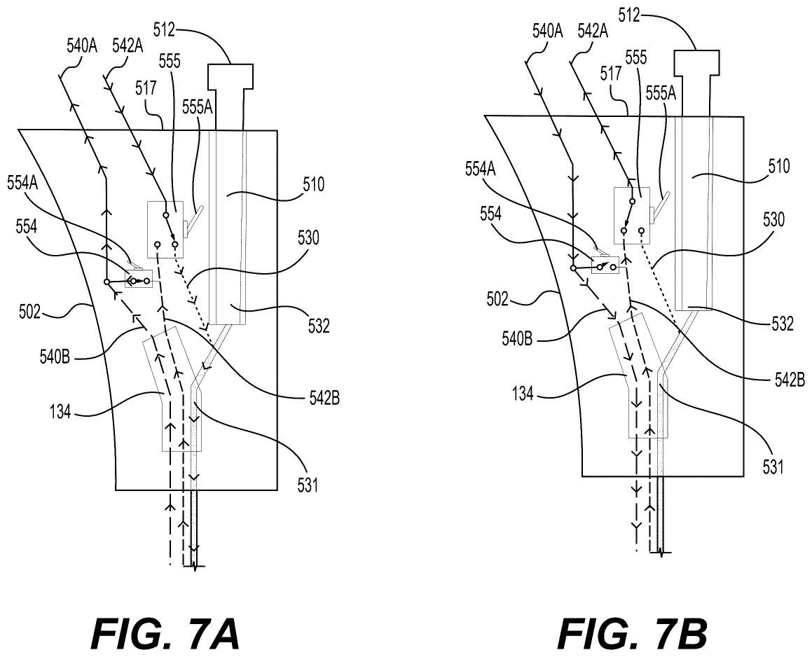

Actuator 554 may include a control switch 554A and actuator 555 may include a control switch 555A. It will be appreciated that reference to “a control switch” is only exemplary, and any suitable actuator mechanism may be used. Each of control switches 554A and 555A may be actuated between at least two positions, which may make or break one or more electrical circuits between one or more distal tip functions (e.g., cauterization and cutting). Actuator 554 may be an on/off type actuator that may electrically connect/disconnect wire 540A with wires 540B or 542B. Actuator 555 may be a single output double throw actuator, which may have a single input (wire 542A) and may have two outputs (wires 542B and 530). Further description of the electrical functionality of actuators 554 and 554 is provided with respect to FIGS. 7A and 7B.

FIG. 7A illustrates handle 502 configured for a first functionality, for example, a cutting functionality. Incoming energy may flow through wire 542A to actuator 555. Actuator 555 may be in a first position, such that energy flows through actuator 555 to wire 530. Actuator 555 being in the first position may also break or disconnect a connection between wire 542A and wire 542B. Wire 530 may be operably (e.g., electrically) connected to cutting electrode 120. Wires 540A/540B and 542A may each act as return wires, and wires 540A/540B and 542B may each be operably connected to cutting electrode 120. Energy may flow from the treatment site through wires 540A/540B to the energy source. Power flowing through wire 542B may flow through actuator 554, which may be in a first position. Power flowing through actuator 554 may then flow along wire 540A back to the energy source or a ground, thereby completing the circuit.

FIG. 7B illustrates handle 502 configured for a second functionality, for example, a cauterization functionality. An operator may switch handle 502 between cutting and cauterization functions by actuating both actuators 554 and 555 from the respective first positions to respective second positions. An energy source operably connected to wire 540A may provide power that bypasses actuator 554 due to actuator 554 being in a second position, breaking the connection between wire 540A and wire 542B through actuator 554. Incoming energy may flow through wires 540A and 540B (which may have a first polarity), which are operably coupled to cauterization electrode 204. In these aspects, energy delivered from cauterization electrode 204 may pass through patient tissue or other portions of the treatment site to cauterization electrode 206. Cauterization electrode 206 may be operably connected to wire 542B. Wire 542B may be operably connected to actuator 555, which may be in a second position, breaking a connection between actuator 555 and wire 530. Energy may flow from cauterization electrode 206, through wire 542B, through actuator 555, and to wire 542A, before flowing to the energy source or a ground, thereby completing the circuit.

A user may actuate actuators (e.g., actuator control switches 554A/554A) 554 and/or 555 to switch handle 502 between a cutting mode and a cauterization mode, and vice-versa. For example, a user may perform a tissue cut with cutting electrode 120 with actuators 554 and 555 in their first positions, respectively. In these aspects, energy may be delivered to tissue via cutting electrode 120, and cauterization electrodes 204 and 206 may help to serve as return electrodes, which may obviate the need for another return electrode (e.g., grounding patch 90). Then, to help stop or reduce bleeding resulting from the tissue cut, a user may wish to use the cauterization function. The user may actuate actuators 554 and 555 to their second positions, respectively, to complete the circuit between cauterization electrodes 204 and 206. In these aspects, energy may be delivered to tissue via one or more cauterization electrodes (e.g., cauterization electrode 204), with one or more cauterization electrodes (e.g., cauterization electrode 206) serving as a return electrode, which may also obviate the need for another return electrode (e.g., grounding patch 90).

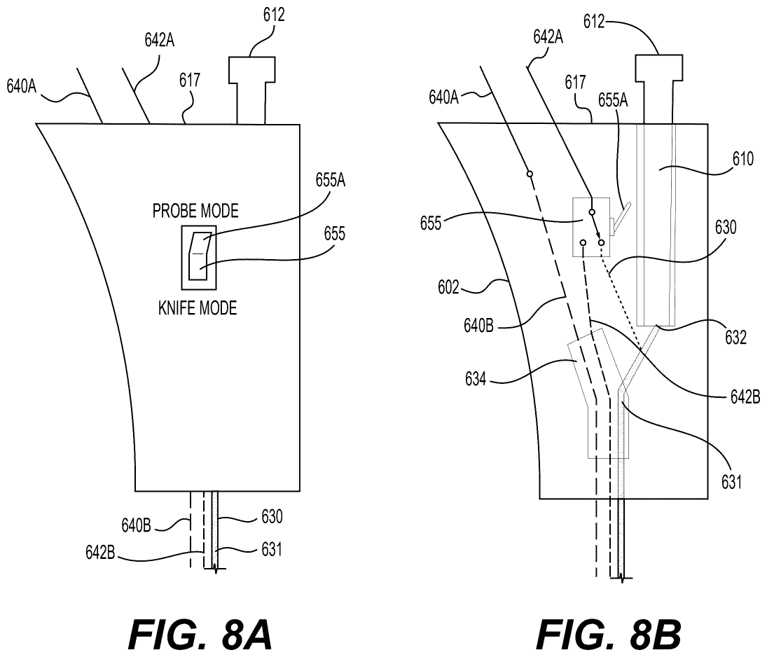

FIG. 8A illustrates an exterior portion of a second alternative handle of the medical device of FIG. 1, and FIG. 8B illustrates a schematic view of the second alternative handle of the medical device of FIG. 1, according to aspects of this disclosure. Handle 602 may be substantially similar to handle 502, with reference numerals having “100” added unless otherwise noted.

Various aspects of this disclosure may use a bipolar configuration for both cutting and cauterization functionality. Handle 602 may include or be coupled a wire 640A and a wire 642A. Handle 602 may include an actuator 655 and actuator control switch 655A, which may be operably connected to both a wire 642A, a wire 642B, and a wire 630. For example, various aspects of this disclosure may use only wires 640A/640B, and not wires 642A/642B, for cutting functionality. Actuator 655 and/or actuator control switch 655A may include one or more labels indicating to a user which mode the medical device is in (e.g., probe/cauterization mode or knife mode). Incoming fluid from port 612 may flow through fluid channel 610 to an opening 632, and fluid may then flow distally though sheath 631 to a distal tip (not shown).

FIGS. 8A and 8B illustrate handle 602 configured for cutting functionality. In this aspect, incoming energy may flow through wire 642A to actuator 655. Actuator 655 may be in a first (e.g., for cauterization) position, such that energy flows through actuator 655 to wire 630. Actuator 655 being in the first position may also break or disconnect a connection between wire 642A and wire 642B. Wire 630 may be operably (e.g., electrically) connected to cutting electrode 120 (FIG. 1). Wire 640A/640B may act as a return wire when using cauterization electrodes 204 and 206, and when using cutting electrode 120. Wire 630 may be enclosed within a sheath 631. Wire 640A/640B may be operably attached to cauterization electrode 204, and wire 642A/642B may be operably attached to cauterization electrode 206.

Wires 640A/640B may be operably connected to cutting electrode 120. Power may flow from cutting electrode 120 along 640B and 640A to the energy source. Power may then flow along wire 640A back to the energy source, thereby completing the circuit.

FIG. 8B illustrates handle 602 configured for cauterization functionality. An operator may switch handle 602 between cutting and cauterization functions by actuating actuator 655 from the first position to the second position. An energy source operably connected to wire 642A may provide power that wire 630 due to actuator 655 being in a second position, breaking the connection between wire 640A and wire 630 through actuator 655. Incoming energy may flow through wires 642A and 642B (which may have a first polarity), which are operably coupled to cauterization electrode 206 and may pass through patient tissue to flow back to the energy source via cauterization electrode 204. Cauterization electrode 204 may be operably connected to wire 642B. Energy may flow from wire 642B to wire 642A, before flowing to the energy source or ground.

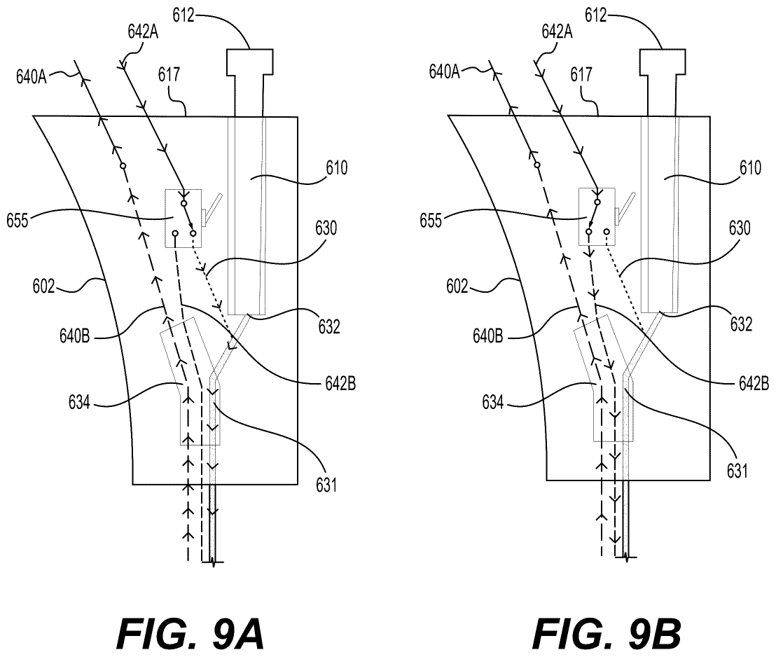

FIG. 9A illustrates handle 602 configured for cutting functionality. Incoming energy may flow through wire 642A to actuator 655. Actuator 655 may be in a first position, such that energy flows through actuator 655 to wire 630. Actuator 655 being in the first position may also break a circuit between wire 642A and wire 642B. Wire 630 may be operably (e.g., electrically) connected to cutting electrode 120. Wires 640A/640B may act as return wires and may each be operably connected to a first electrode, for example, cutting electrode 120. Power may flow from cutting electrode 120 along wires 640A/640B to the energy source. Power flowing through wire 642B may flow through actuator 554, which may be in a first position. Power flowing through actuator 655 may then flow along wire 640A back to the energy source, thereby completing the circuit.

FIG. 9B illustrates handle 602 configured for cauterization functionality. An operator may switch handle 602 between cutting and cauterization functions by actuating actuator 655 from it first position to its second position. Incoming energy may flow through wires 640A and 640B (which may have a first polarity), which are operably coupled to cauterization electrode 204 and may pass through patient tissue to flow back to the energy source via cauterization electrode 206. Cauterization electrode 206 may be operably connected to wire 642B. Wire 642B may be operably connected to actuator 655, which may be in a second position, breaking a connection between actuator 655 and wire 630. Energy may flow through actuator 655 to wire 642A, before flowing to the energy source, thereby completing the circuit.

A user may actuate actuator 655 (e.g., actuator control switch 655A) to switch handle 602 between cutting and cauterization and vice-versa. For example, a user may perform a tissue cut with cutting electrode 120 with actuator 655 in its first position. To help stop or reduce bleeding resulting from the tissue cut, a user may wish to use the cauterization function. The user may actuate actuator 655 to its second position to complete the circuit between cauterization electrodes 204 and 206 (e.g., via energy traveling from cauterization electrode 204 through patient tissue to cauterization electrode 206).

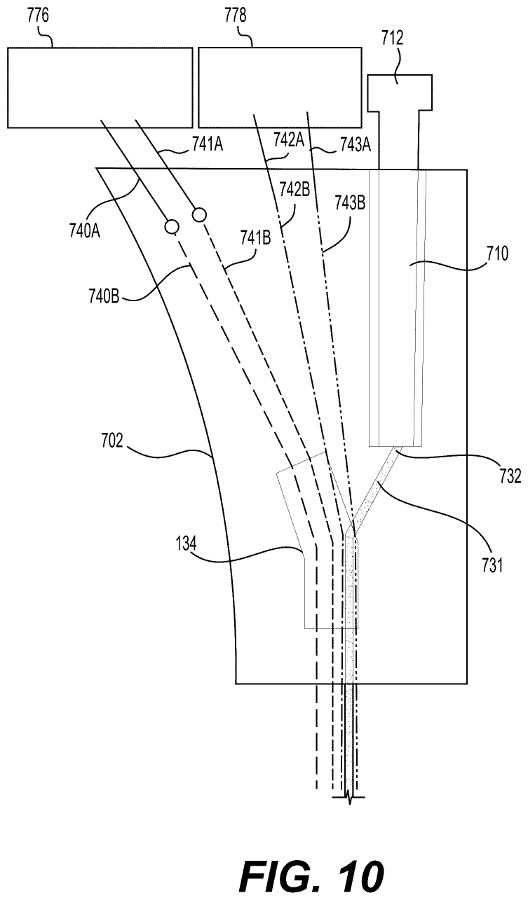

FIG. 10 illustrates a handle 702, according to another aspect of the disclosure. Handle 702 may include a cauterization energy source 776 and a cutting energy source 778. Both the cauterization and cutting functions of handle 702 may be bipolar, and the cauterization and cutting functions of handle 702 may each rely on separate power sources. Incoming fluid from port 712 may flow through fluid channel 710 to an opening 732, and fluid may then flow distally though sheath 731 to a distal tip (not shown). Power may flow from cauterization energy source 776 to a wire 740A, a wire 740B, and cauterization electrode 204 (FIG. 1).

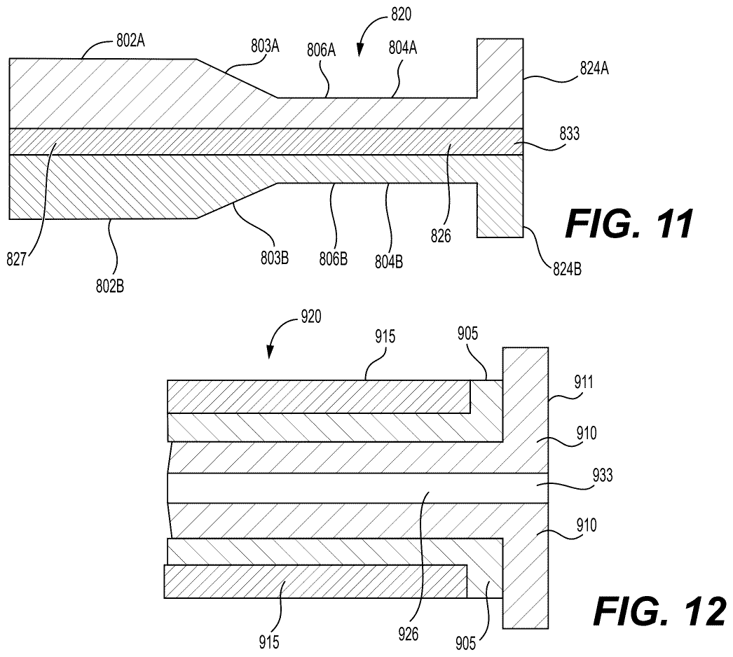

Power may flow through patient tissue to cauterization electrode 206 (FIG. 1), to a wire 741B, a wire 741A, and then to cauterization energy source 776, thereby completing the circuit for cauterization functionality. In some aspects, fluid may be delivered through a lumen 826 and an opening 833 via a port 712, which is shown with respect to FIG. 11. In some aspects, fluid may be delivered through a lumen 926 and opening 933 via port 712, which is shown with respect to FIG. 12. For cutting functionality, power may flow from cutting energy source 778 to a wire 742A, a wire 742B, and then to at least one portion of cutting electrode 820.

Power may flow from at least another portion of cutting electrode 820 through wire 743B, 743A to cutting energy source 778, thereby completing the circuit for cutting functionality. It will be appreciated that cauterization energy source 776 and cutting energy source 778 may each be independently energized, activated, or otherwise turned on or off (e.g., connecting or disconnecting the corresponding circuit). Thus, cauterization electrodes 204/206 and cutting electrode 120 may all be active at the same time. Alternatively, only electrodes 204/206 may be active, or only cutting electrode 120 may be active, for example, based on which of cauterization energy source 776 or cutting energy source 778 is active.

FIG. 11 illustrates a cross-sectional view of a knife electrode 820, according to another aspect of the disclosure. Knife electrode 820 may be compatible with handle 702, for example, coupled to wires 742B and 743B. Knife electrode 820 may replace cutting electrode 120 in scenarios in which there are two energy sources (e.g., cauterization energy source 776 and cutting energy source 778). Knife electrode 820 may include a first portion or side 806A including a proximal portion 802A, an intermediate portion 803A, a distal portion 804A. Intermediate portion 803A may be sloped or tapered, for example, between proximal portion 802A and distal portion 804A.

Distal portion 804A may have a smaller diameter than proximal portion 802A.

Additionally, first side 806A may have a distal tip 824A, which may include a larger diameter than distal portion 804A.

Knife electrode 820 may include a second portion or side 806B including proximal portion 802B, a sloped middle portion 803B, a distal portion 804B that may have a smaller diameter than proximal portion 802B, and a distal tip 824B. An electrically insulative material 827 may separate first portion 806A from second portion 806B. First side 806A and second side 806B may thus be electrically isolated from one another, and may have opposite polarities. Power from cutting energy source 778 may flow through wires 742A and 742B to first side 806A, through tissue (which may close the circuit between first side 806A and second side 806B) to second side 806B, and then to wires 743B and 743A back to cutting energy source 778, thereby completing the circuit.

FIG. 12 illustrates a cross-sectional view of another knife electrode 920, according to additional aspects of the disclosure. Knife electrode 920 may be compatible with handle 702, for example, coupled to wires 742B and 743B. Knife electrode 920 may replace cutting electrode 120 in scenarios in which there are two energy sources (e.g., cauterization energy source 776 and cutting energy source 778). Fluid may be delivered through lumen 926 and opening 933 via port 712. Knife electrode 920 may include a first conductive portion 910, a second conductive portion 915, and an insulative portion 905 disposed between first conductive portion 910 and second conductive portion 915. First conductive portion 910 may be operably connected to wires 742A and 742B (having a first polarity). First conductive portion may include a distal portion or protrusion 911. Distal portion 911 may have a greater diameter than second conductive portion 915. Second conductive portion 915 may be operably connected to wires 743A and 743B (having a second polarity). Energy may flow from cutting energy source 778 to wires 742A and 742B to first conductive portion 910. Energy may flow through patient tissue as part of a medical procedure, and may then flow from second conductive portion 915, along wires 743B and 743A, and to cutting energy source 778, thereby completing the circuit.

The medical devices and methods discussed above may help to allow a user to treat tissue by delivering electrical energy into the tissue. The medical device and methods discussed above may also help to allow a user to treat tissue by delivering fluid, either simultaneously or sequentially with the energy delivery. For example, a user may couple an electrode (e.g., a cutting or knife electrode) to the distal end and deliver the distal end to an interior lumen of a subject to deliver medical therapy in a portion of a procedure (e.g., mark, cauterize, or resect tissue). The user may also deliver fluid distally out of the distal end of the electrode, either simultaneously or sequentially with the energy delivered, which may help the user to more quickly and efficiently deliver the medical therapy, for example, cut, dissect, ablate, mark, coagulate, cauterize, or otherwise treat tissue. Moreover, the user may deliver fluid and energy without removing the medical device from the patient or subject, which may help to reduce the costs and duration of the procedure, also potentially reducing the risks to the subject. The disclosed helical electrodes can be used for both cauterization and as returns for a cutting electrode. In some aspects, the helical electrodes are spaced apart, such that energy may flow through tissue (e.g., for cauterization) when passing from one electrode to another.

While principles of this disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein.

Accordingly, the disclosure is not to be considered as limited by the foregoing description.

We claim:

1. A medical device for delivering energy and/or fluid, the medical device comprising:
a handle including:
one or more electrical connections configured to electrically connect the medical device to an energy source; and
a fluid port configured to receive fluid from a fluid source;
a shaft including a lumen for transmitting the fluid from the fluid source and one or more conductors for transmitting energy from the energy source, the one or more conductors including a first conductor, a second conductor, and a third conductor;
a first electrode at a distal end of the shaft, wherein the first electrode is electrically coupled to the first conductor to deliver energy from the energy source to the first electrode, wherein the first electrode includes a lumen having a distal opening for delivering fluid from the fluid source to the distal opening; and
a second electrode and a third electrode disposed on an outer surface of the distal end of the shaft, wherein the second electrode is electrically coupled to the second conductor, and wherein the third electrode is electrically coupled to the third conductor to deliver energy from the energy source to the third electrode.

2. The medical device of claim 1, wherein a hub comprises a first electrical connection of the one or more electrical connections, wherein the shaft includes a sheath, wherein the sheath defines the lumen.

3. The medical device of claim 2, further comprising:
a ground configured to be coupled to a patient, wherein the ground is electrically coupled to the first conductor.

4. The medical device of claim 1, wherein the second conductor and the third conductor have opposite polarities.

5. The medical device of claim 2, wherein the hub is coupled to the fluid port.

6. The medical device of claim 2, wherein the handle further comprises a handle body that defines a channel, wherein the hub is movably coupled to the handle body, wherein the hub is configured to move in the channel.

7. The medical device of claim 1, further comprising:
a first actuator electrically coupled to the second conductor and the third conductor; and
a second actuator electrically coupled to the first conductor and third conductor,
wherein, in a first configuration of the first actuator and the second actuator, the energy source is electrically connected to the first electrode, and the energy source is not electrically connected to the second electrode and the third electrode,
wherein the second conductor and the third conductor comprise, respectively, a first electrical connection of the one or more electrical connections and a second electrical connection of the one or more electrical connections.

8. The medical device of claim 7, wherein the first conductor has an opposite polarity of the second conductor and the third conductor.

9. The medical device of claim 7, wherein, in a second configuration of the first actuator and the second actuator, the energy source is not electrically connected to the first electrode, and the energy source is electrically connected to the second electrode and the third electrode.

10. The medical device of claim 9, wherein the second conductor and the third conductor have opposite polarities.

11. The medical device of claim 1, further comprising:
an actuator electrically coupled to the first conductor and the third conductor,
wherein, in a first configuration of the actuator, the energy source is electrically connected to the first electrode, and the energy source is not electrically connected to the second electrode and the third electrode,
wherein, in a second configuration of the actuator, the energy source is not electrically connected to the first electrode, and the energy source is electrically connected to the second electrode and the third electrode,
wherein the second conductor and the third conductor comprise, respectively, a first electrical connection of the one or more electrical connections and a second electrical connection of the one or more electrical connections.

12. The medical device of claim 1, wherein the energy source is a first energy source, wherein the one or more conductors further include a fourth conductor, wherein the one or more electrical connections include the first conductor, the second conductor, the third conductor, and the fourth conductor, further comprising:
a second energy source, wherein the second conductor and the third conductor are electrically connected to the first energy source, wherein the first conductor and the fourth conductor are electrically connected to the second energy source.

13. The medical device of claim 1, wherein the first electrode is configured to be advanced distally and retracted proximally relative to the distal end of the shaft, wherein the first electrode includes a shaft portion and a widened distal portion that is wider than the shaft portion.

14. The medical device of claim 12, wherein the second conductor and the third conductor have opposite polarities, wherein the first conductor and the fourth conductor have opposite polarities.

15. The medical device of claim 1, wherein the second electrode and the third electrode extend helically around the distal end of the shaft, wherein the second electrode and the third electrode are spaced apart.

16. A medical device for delivering energy, the medical device comprising:
a handle including:
one or more electrical connections configured to electrically connect the medical device to an energy source; and
a shaft including a lumen and a sheath, wherein the sheath defines the lumen;
one or more conductors for transmitting energy from the energy source, wherein the sheath is electrically insulative;
a first electrode at a distal end of the shaft, comprising:
a first portion electrically coupled to a first conductor of the one more conductors and positioned on a first side of the sheath; and
a second portion electrically coupled to a second conductor of the one or more conductors and positioned on a second side of the sheath; and
a second electrode disposed on an outer surface of the distal end of the shaft, wherein the second electrode is electrically coupled to a third conductor of the one or more conductors; and
a third electrode disposed on the outer surface of the distal end of the shaft, wherein the third electrode is electrically coupled to a fourth conductor of the one or more conductors.

17. The medical device of claim 16, wherein the first electrode comprises a cutting electrode, and wherein the second electrode and the third electrode comprise cauterization electrodes.

18. The medical device of claim 16, wherein the first conductor and the second conductor have opposite polarities.

19. A medical device for delivering energy and/or fluid, the medical device comprising:
a handle including:
one or more electrical connections configured to electrically connect the medical device to an energy source; and
a fluid port configured to receive fluid from a fluid source;
a shaft including a lumen and a sheath, wherein the sheath defines the lumen for transmitting the fluid from the fluid source and one or more conductors for transmitting energy from the energy source, wherein the sheath is electrically insulative;
a first electrode at a distal end of the shaft, comprising:
a first portion electrically coupled to a first conductor of the one more conductors and disposed radially outward of the sheath;
a second portion electrically coupled to a second conductor of the one or more conductors and disposed radially outward of the first portion;
an electrical insulator disposed between the first portion and the second portion;
an electrode lumen extending through a portion of the first electrode, wherein the electrode lumen is fluidly connected to the lumen in the shaft, and wherein the electrode lumen includes a distal opening for delivering fluid from the fluid source;
a second electrode disposed on an outer surface of the distal end of the shaft; and
a third electrode disposed on the outer surface of the distal end of the shaft, wherein the second electrode is electrically coupled to a third conductor of the one or more conductors, and wherein the third electrode is electrically coupled to a fourth conductor of the one or more conductors.

20. The medical device of claim 19, wherein the first portion further comprises a distal protrusion, wherein the distal protrusion includes a larger diameter than the second portion.
